(19) United States
(12) Patent Application Publication
(10) Pub. No.: US 2014/0243412 A1
(43) Pub. Date: Aug. 28, 2014

(54) PROCESS FOR PREPARATION OF PREGABALIN

(71) Applicant: Dr. Reddy's Laboratories Ltd., Hyderabad (IN)

(72) Inventors: Srinivas GANGULA, Karimnagar (IN); Uday Kumar NEELAM, Hyderabad (IN); Krishna VINIGARI, Mahaboobnagar (IN); Vilas Hareshwar DAHANUKAR, Hyderabad (IN); Syam Kumar Unniaran KUNHIMON, Hyderabad (IN); Srinivas Reddy GADE, Hyderabad (IN)

(73) Assignee: Dr. Reddy's Laboratories Ltd., Hyderabad (IN)

(21) Appl. No.: 14/192,032

(22) Filed: Feb. 27, 2014

(30) Foreign Application Priority Data
Feb. 28, 2013 (IN) 872/CHE/2013
Sep. 25, 2013 (IN) 4332/CHE/2013

(51) Int. Cl. C07C 227/08; C07C 231/02

(52) U.S. Cl. CPC C07C 227/08; C07C 231/02
USPC 514/561; 562/553

(57) ABSTRACT

Aspects of the present invention relate to improved process for preparation of (R)(−)-3-(carbamoylmethyl)-5-methylhexanoic acid (R-CMHA) of the formula (II) or its pharmaceutically acceptable salts in the presence of a lewis acid, process for preparation of pregabalin using R-CMHA of the formula (II) or its pharmaceutically acceptable salts prepared according to the present invention and process for preparation of pregabalin with low amount of undesired impurity.

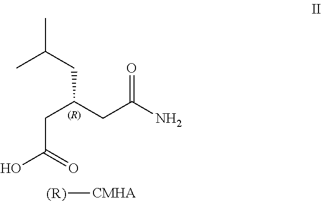

This application claims priority to Indian provisional application Nos. 872/CHE/2013 filed on Feb. 28, 2013; and 4332/CHE/2013 filed on Sep. 25, 2013, all of which are hereby incorporated by reference in their entireties.

### INTRODUCTION

Aspects of the present invention relate to improved process for preparation of (R)(−)-3-(carbamoylmethyl)-5-methylhexanoic acid (R-CMHA) of the formula (II) or its pharmaceutically acceptable salts in the presence of a lewis acid and process for preparation of pregabalin using (R)(−)-3-(carbamoylmethyl)-5-methylhexanoic acid (R-CMHA) of the formula (II) or its pharmaceutically acceptable salts prepared according to the present invention.

Another aspect of the present invention relate to process for preparing pregabalin with low amount of undesired impurity.

### BACKGROUND

The drug compound having the adopted name “Pregabalin” has chemical name: (S)-(+)-3-Aminomethyl-5-methyl hexanoic and is represented by structure of formula I.

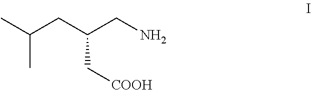

Pregabalin is a gamma.-amino butyric acid or (S)-3-isobutyl (GABA) analogue. (S)-Pregabalin has been found to activate GAD (L-glutamic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brains synapses. (S)-Pregabalin has analgesic, anticonvulsant, and anxiolytic activity. Preparation of (S)-Pregabalin is disclosed in U.S. Pat. No. 5,563,175, U.S. Pat. No. 6,197,819B1, U.S. Pat. No. 5,616,793, Drugs of Future, 24 (8), 862-870 (1999) and in many other prior references.

Several patents and published patent applications for example U.S. Pat. No. 5,616,793, WO 2006/122258, WO 2006/122255 and WO 2006/121557 disclose a more convenient preparation of pregabalin by means of a Hofmann rearrangement of (R)-(−)-3-(carbamoylmethyl)-5-methylhexanoic acid, a compound of formula II.

U.S. Pat. No. 8,071,808B2 provides a process for preparing pregabalin of formula I substantially free of impurities by reacting (R)-(−)-3-(carbamoylmethyl)-5-methylhexanoic acid of formula II with sodium hypochlorite at a temperature 50-70 degree Celsius.

### SUMMARY

First aspect of the present application provides process for preparation of (R)(−)-3-(carbamoylmethyl)-5-methylhexanoic acid (R-CMHA) of formula (II) or its pharmaceutically acceptable salts which comprises:
a) reacting an ester of formula (III) with ammonia reagent in presence of a lewis acid and a suitable solvent;

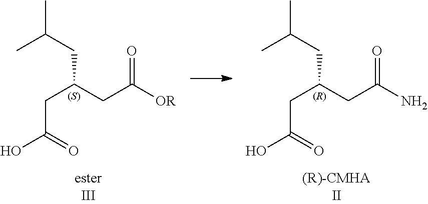

wherein R═C₁-C₆ alkyl, aryl, alkyl aryl
b) optionally isolating compound of formula (II);
c) optionally converting compound of formula (II) to its pharmaceutically acceptable salts;
d) optionally purifying compound of formula (II) or its pharmaceutically acceptable salts.

Second aspect of the present application provides process for preparation of pregabalin

which comprises:
a) reacting an ester of formula (III) with ammonia reagent in presence of a lewis acid and a suitable solvent to provide (R)(−)-3-(carbamoylmethyl)-5-methylhexanoic acid (R-CMHA) of the formula (II) or its pharmaceutically acceptable salts;
b) reacting (R)(−)-3-(carbamoylmethyl)-5-methylhexanoic acid (R-CMHA) of formula (II) or its pharmaceutically acceptable salts with a hypohalite in presence of suitable base and a solvent to provide pregabalin of formula (I);
c) isolating pregabalin of formula (I);
d) optionally purifying pregabalin in a suitable solvent.

Third aspect of the present application provides process for preparation of pregabalin substantially free of undesired 4-ene impurity of formula IV

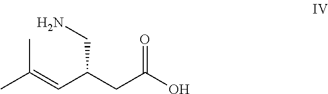

which comprises
a) reacting (R)(−)-3-(carbamoylmethyl)-5-methylhexanoic acid (R-CMHA) or a salt of the formula II with hypochlorite in presence of suitable base and solvent;

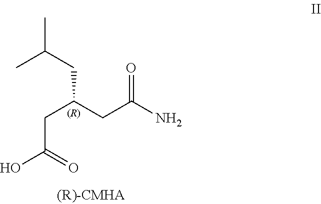

b) treating the reaction mass with an acid to get pH about 2.0-3.0;
c) treating with suitable base to result the pH of the mass about 5.0-5.5;
d) isolating of pregabalin;
e) optionally purifying pregabalin in a suitable solvent.

Fourth aspect of the present application provides pharmaceutical compositions comprising pregabalin of formula (I) or its pharmaceutically acceptable salts prepared according to process of the present application together with one or more pharmaceutically acceptable excipient, carrier and diluents.

### DETAILED DESCRIPTION

First aspect of the present application provides process for preparation of (R)(−)-3-(carbamoylmethyl)-5-methylhexanoic acid (R-CMHA) or a salt of formula (II) which comprises:
a) reacting an ester of formula (III) with ammonia reagent in presence of a lewis acid and a suitable solvent;

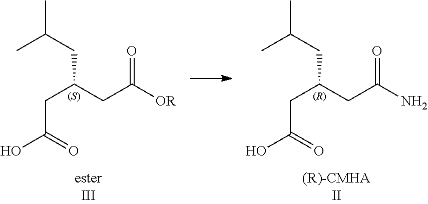

wherein R═C₁-C₆ alkyl, aryl, alkyl aryl
b) optionally isolating compound of formula (II);
c) optionally converting compound of formula (II) to its pharmaceutically acceptable salts;
d) optionally purifying compound of formula (II) or its pharmaceutically acceptable salts.

The suitable esters that may be used in step a) include but are not limited to methyl, ethyl, isopropyl, butyl, benzyl, phenyl and the like.

In one of the preferred embodiments, the ester may be prepared by following enzymatic reactions known in the art. The enzymes that may be used for preparation of ester of compound of formula (II) include, but are not limited to lipase, esterase, protease and the like.

The present invention also includes process for preparation of ester of formula (III) by known chemical methods.

The suitable ammonia reagents that may be used in step (a) include, but are not limited to NH₃ gas, (NH₃)₂CO₃, HCONH₂, Aq.NH₃, NH₂CONH₂, HCOONH₂, NH₄Cl, ammonium chloride and the like.

The suitable Lewis acid that may be used in step (a) include, but are not limited to calcium chloride, lithium chloride, cerium chloride, Indium chloride, magnesium chloride, magnesium bromide, ammonium chloride, zinc chloride, boron trifluoride dietherate complex or the like.

Step (a) may be carried out in one or more suitable solvents. Suitable solvents that may be used in step (a) include, but are not limited to alcohol solvents, such as, for example, methanol, ethanol, propanol, 1-propanol, 2-propanol, butanol, pentanol or ethylene glycol or glycerol, or the like; aliphatic or alicyclic hydrocarbon solvents, such as, for example, hexane, heptane, pentane, cyclohexane, methylcyclohexane, or the like; halogenated hydrocarbon solvents, such as, for example, dichloromethane, chloroform, 1,1,2-trichloroethane, 1,2-dichloroethene, or the like; aromatic hydrocarbon solvents, such as, for example, toluene, xylene, chlorobenzene, tetralin, or the like; or any mixtures thereof.

The temperature at which step (a) may be carried out in between about 0° C. and about 100° C., preferably between about 20° C. and about 60° C.

Isolation and purification can be further carried out if desired in step (b), (c) and (d), by any suitable separation or purification procedure such as, for example, filtration, centrifugation, extraction, crystallization, conventional isolation and refining means such as concentration, concentration under reduced pressure, solvent-extraction, crystallization and phase-transfer methods.

The inorganic base used for conversion of (R)(−)-3-(carbamoylmethyl)-5-methylhexanoic acid (R-CMHA) of formula (II) to its pharmaceutically acceptable salts include, but are not limited to sodium hydroxide, lithium hydroxide, potassium hydroxide and the like.

Second aspect of the present application provides process for preparation of pregabalin

which comprises:
a) reacting an ester of formula (III) with ammonia reagent in presence of a lewis acid and a suitable solvent to provide (R)(−)-3-(carbamoylmethyl)-5-methylhexanoic acid (R-CMHA) of formula (II) or its pharmaceutically acceptable salts;
b) reacting (R)(−)-3-(carbamoylmethyl)-5-methylhexanoic acid (R-CMHA) of the formula (II) or its pharmaceutically acceptable salts with a hypohalite in presence of suitable base and a solvent to provide pregabalin of formula (I);
c) isolating pregabalin of formula (I);
d) optionally purifying pregabalin in a suitable solvent.

The reagents and solvents for step (a) may be selected from one or more suitable reagents and solvents as described in step (a) of the first embodiment.

Step (b) may be carried out in presence of suitable hypochlorite. Suitable hypochlorite that may be used in step (b) include, but are not limited to sodium hypochlorite, sodium hypobromite, calcium hypochlorite, calcium hypobromite or the like.

Step (b) may be carried out in the presence of one or more suitable bases. Suitable bases that may be used in step (a) include, but are not limited to alkali metal hydroxides, such as, for example, lithium hydroxide, sodium hydroxide, potassium hydroxide, and cesium hydroxide; alkaline earth metal hydroxides, such as, for example, barium hydroxide, strontium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, sodium bicarbonate, potassium bicarbonate, or the like.

Step (b) may be carried out in one or more suitable solvents. Suitable solvents that may be used in step (a) include, but are not limited to water or mixture of water and suitable organic solvent. Suitable organic solvents that may be used include, but are not limited to alcohol solvents, such as, for example, methanol, ethanol, propanol, 1-propanol, 2-propanol, butanol, pentanol, ethylene glycol, glycerol, or the like; aliphatic or alicyclic hydrocarbon solvents, such as, for example, hexane, heptane, pentane, cyclohexane, methylcyclohexane, or the like; halogenated hydrocarbon solvents, such as, for example, dichloromethane, chloroform, 1,1,2-trichloroethane, 1,2-dichloroethene, or the like; aromatic hydrocarbon solvents, such as, for example, toluene, xylene, chlorobenzene, tetralin, or the like; or any mixtures thereof.

The temperature at which step (b) may be carried out in between about 0° C. and about 100° C., preferably between about 5° C. and about 48° C.

Isolation of the pregabalin can be further carried out if desired in step (c), by any suitable separation or purification procedure such as, for example, filtration, centrifugation, extraction, crystallization, conventional isolation and refining means such as concentration, concentration under reduced pressure, solvent-extraction, crystallization and phase-transfer methods.

The pregabalin obtained after step (c) of the present invention may be optionally further purified to get the ICH grade material of API in order to use the same for finished dosage forms.

Purification in step (d) may be carried out in one or more suitable solvents. Suitable solvents that may be used in step (d) include, but are not limited to water or mixture of water and organic solvent. Suitable organic solvents that may be used include, but are not limited to alcohol solvents, such as, for example, methanol, ethanol, isopropanol, butanol, pentanol, ethylene glycol, glycerol, or the like; or any mixtures thereof.

The temperature at which step (d) may be carried out in between about 0° C. and about 100° C., preferably between about 5° C. and about 75° C.

The processes of the present invention may also include isolation of individual intermediate or processed for further steps without isolation of intermediates wherever applicable.

The process of the present application is not only environment friendly and also provides better yield with required chiral purity. The process is further suitable to practice at on industrial scale. The calcium chloride mediated process not only covert the ester to amide but also convert the potential trans esterification product to R-CMHA.

In the synthesis of pregabalin by carrying out Hoffmann reaction, the formation of ˜0.51 RRT impurity was observed and which did not have wash ability in the subsequent conventional purification processes. The formation of pregabalin starting from (R)-(−)-3-(carbamoylmethyl)-5-methylhexanoic acid of formula II under Hoffmann reaction conditions is represented as follows:

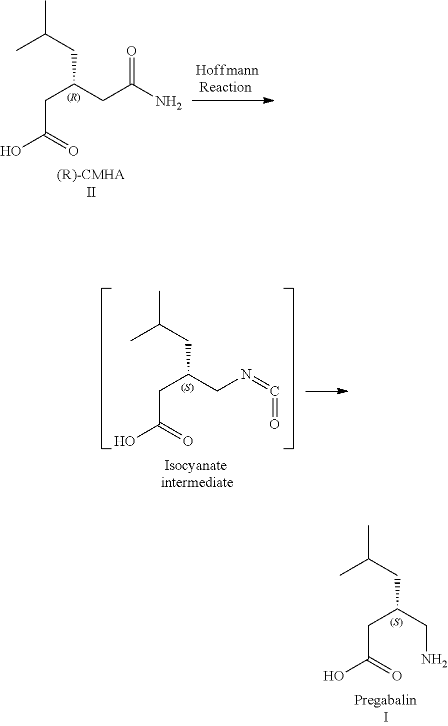

The pregabalin obtained as per the above reaction conditions by following the process disclosed in prior references is having an unknown impurity above the desired levels and was unable to wash the impurity during recrystallization from a solvent. Based on LC-MS analysis this impurity was characterized as 4-ene impurity of pregabalin which is represented by formula IV. This impurity has identified at RRT 0.51 in HPLC analysis.

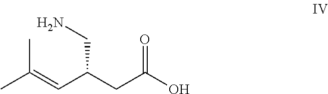

Therefore, it is required to control the ˜0.51 RRT impurity to a desired level within the process of Hoffmann reaction or during work up process. It has become essential to adjust the pH of the reaction mass at about 2.0-3.0 with an acid followed by raising the pH to about 5.0-5.5 to control the impurity to a desired level after completion of Hoffmann reaction.

Third aspect of the present application provides process for preparation of pregabalin substantially free of undesired 4-ene impurity of formula IV,

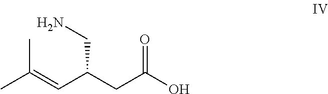

which comprises
a) reacting (R)(−)-3-(carbamoylmethyl)-5-methylhexanoic acid (R-CMHA) or a salt of the formula II with hypochlorite in presence of suitable base and solvent;

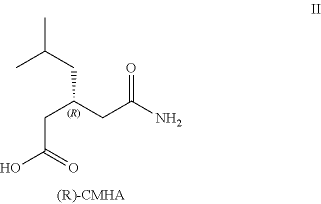

b) treating the reaction mass with an acid to get pH about 2.0-3.0;
c) treating with suitable base to result the pH of the mass about 5.0-5.5;
d) isolating of pregabalin;
e) optionally purifying pregabalin in a suitable solvent.

The hypochlorite, base and solvent for step a) may be selected from one or more suitable hypochlorite, base and solvent as described in step b) of second aspect of the present application.

R)(−)-3-(carbamoylmethyl)-5-methylhexanoic acid or a salt of the formula II can be prepared according to the present invention or methods known in prior art. RCMHA may be obtained through chemical synthesis or by enzymatic reaction.

The temperature at which step (a) may be carried out in between about 0° C. and about 100° C., preferably between about 5° C. and about 48° C.

In order to control the impurity at-0.51 RRT (4-ene impurity), the pH of reaction mass after completion of step (a) was adjusted to 2-3 by using a suitable acid in step (b).

The suitable acids that may be used in step (b) include, but are not limited to mineral acids such as hydrochloric acid, hydrobromic acid, or other acids such as sulfuric acid, nitric acid, phosphoric acid, boric acid or the like; organic acids such as acetic acid, formic acid, methane sulfonic acid, lactic acid, citric acid, oxalic acid, uric acid or the like. The pH adjustment may also done using gaseous acids such as HCl gas.

The temperature at which step (b) may be carried out in between about 0° C. and about 40° C., preferably between about 5° C. and about 35° C.

Further re-adjusting the pH of the reaction mass from 2.0-3.0 to 5.0-5.5 in step (c) was carried out using a base.

The suitable bases that may be used in step (c) include, but are not limited to alkali metal hydroxides, such as, for example, lithium hydroxide, sodium hydroxide, potassium hydroxide, and cesium hydroxide; alkaline earth metal hydroxides, such as, for example, barium hydroxide, strontium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, sodium bicarbonate, potassium bicarbonate, or the like.

The temperature at which step (c) may be carried out in between about 0° C. and about 40° C., preferably between about 5° C. and about 35° C.

Isolation of the pregabalin can be further carried out if desired in step (d), by any suitable separation or purification procedure such as, for example, filtration, centrifugation, extraction, crystallization, conventional isolation and refining means such as concentration, concentration under reduced pressure, solvent-extraction, crystallization, phase-transfer.

The pregabalin obtained in the step (d) is having the purity in the range of 99% to 99.9% and the content of 4-ene impurity of pregabalin is below 0.15% w/w, preferably less than 0.05% and more preferably in “not detected” level in HPLC analysis.

The pregabalin obtained after step (d) of the present invention may be optionally further purified to get the ICH grade material of API in order to use the same for finished dosage forms in step (e).

Purification in step (e) may be carried out in one or more suitable solvents. Suitable solvents that may be used in step (e) include, but are not limited to water or mixture of water and organic solvent. Suitable organic solvents that may be used include, but are not limited to alcohol solvents, such as, for example, methanol, ethanol, propanol, 1-propanol, 2-propanol, butanol, pentanol, ethylene glycol, glycerol, or the like; aliphatic or alicyclic hydrocarbon solvents, such as, for example, hexane, heptane, pentane, cyclohexane, methylcyclohexane, or the like; halogenated hydrocarbon solvents, such as, for example, dichloromethane, chloroform, 1,1,2-trichloroethane, 1,2-dichloroethene, or the like; aromatic hydrocarbon solvents, such as, for example, toluene, xylene, chlorobenzene, tetralin, or the like; or any mixtures thereof.

The temperature at which step (e) may be carried out in between about 0° C. and about 100° C., preferably between about 5° C. and about 75° C.

During the isolation of the crude pregabalin most of the process related impurities were washed out in mother liquor and where as the 4-ene impurity is removed by adjusting the pH acidic. The following table shows comparison of the content of 4-ene impurity at different pH levels (Table-1).

TABLE 1
Effect of acidic pH adjustment (pH: 2-3) on
content of 4-ene impurity

| pH range | Content of impurity (%) |
|---|---|
| 13.5-14.0 (as such reaction mass) | 0.39 |
| Direct Isolation at pH about 5.0 | 0.14 |
| Direct Isolation at pH about 6.0 | 0.16 |
| Adjusting to pH about 2-3 and | Not detected |
| followed raising to about 5.0-5.5 | |

The processes of the present invention may also include isolation of individual intermediate or processed for further steps without isolation of intermediates wherever applicable.

The term substantially free means the content of 4-ene impurity in pregabalin is below 0.15% w/w, preferably less than 0.05% and more preferably in “not detected”.

The HPLC conditions described herein is used to ascertain the purity of pregabalin, including analyzing pregabalin produced through present invention for the presence of impurity which has a HPLC relative retention time of ˜0.51. A two-component mobile phase (A:B) is prepared by mixing mobile phase A (100% degassed buffer) and mobile phase B (water:acetonitrile, 25:75, v/v).

### Typical Chromatographic Conditions:

| Column | X-Bridge C18 150 × 4.6 mm, 3.5 m | | |
|---|---|---|---|
| Flow rate | 0.8 ml/min | | |
| Column oven | 25° C. | | |
| temperature | | | |
| Wave length | 210 nm | | |
| Injection Volume | 15 μL | | |
| Run time | 60 minutes. | | |
| Auto sampler | 5.0° C. ± 2° C. | | |
| temperature | | | |
| Diluent | Water: Acetonitrile: Methanol 8:1:1 ratio | | |
| GRADIENT PROGRAM | Time | % Mobile | % Mobile |
| | (min) | Phase-A | Phase-B |
| | 0.00 | 97 | 3 |
| | 35.0 | 30 | 70 |
| | 45.0 | 30 | 70 |
| | 45.01 | 97 | 3 |
| | 60.0 | 97 | 3 |

The process of the present application is not only environment friendly and also provides better yield with required purity. The process is further suitable to practice at on industrial scale.

Yet another aspect of the present application provides pharmaceutical compositions comprising pregabalin of formula (I) or its pharmaceutically acceptable salts prepared according to process of the present application together with one or more pharmaceutically acceptable excipient, carrier and diluents.

Pharmaceutically acceptable excipients that find use in the present disclosure include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

### Definitions

The following definitions are used in connection with the present invention unless the context indicates otherwise.

“HPLC or High-performance liquid chromatography” is a chromatographic technique used to separate a mixture of compounds with the purpose of identifying, quantifying or purifying the individual components of the mixture.

“LC-MS or Liquid chromatography-mass spectrometry” is a chemistry technique that combines the physical separation capabilities of liquid chromatography (or HPLC) with the mass analysis capabilities of mass spectrometry.

“Hofmann reaction” is the organic reaction of a primary amide to a primary amine with one fewer carbon atom.

A “Lewis acid” is an electron-pair acceptor and therefore able to react with a Lewis base to form a Lewis adduct, by sharing the electron pair furnished by the Lewis base.

An “alcohol solvent” is an organic solvent containing a carbon bound to a hydroxyl group. “Alcoholic solvents” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆alcohols, or the like.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

Certain specific aspects and embodiments of the present invention will be explained in more detail with reference to the following examples, which are provided for purposes of illustration only and should not be construed as limiting the scope of the present invention in any manner.

### EXAMPLES

### Example 1

### Preparation of (R)(−)-3-(carbamoylmethyl)-5-methylhexanoic acid (R-CMHA) (II)

(S)-3-(2-ethoxy-2-oxoethyl)-5-methylhexanoic acid (50 gm, 0.23 moles) (II) was dissolved in methanol (600 ml). Calcium chloride (38 gm, 0.345 moles) was added to the reaction mixture and pressurized with dry NH₃ gas to 4.0 to 4.5 kg/cm² in an auto clave vessel. The reaction mass was heated to 55-60° C. for 10-12 hrs. After completion of the reaction, the reaction mass was distilled at 45° C. Water (175 ml) was added to the solid suspension and pH was adjusted to 3.0 by adding conc.HCl solution (20 ml) at 5° C. under stirring. The solid mass was stirred at 0-5° C. for 2-3 hr. Solid was filtered, washed with water (25 ml) and dried under vacuum to give the title compound.

Yield: 39 g (90%)

HPLC: 88.92%

### Example 2

### Purification of (R)(−)-3-(carbamoylmethyl)-5-methylhexanoic acid (R-CMHA) (II)

(R)(−)-3-(carbamoylmethyl)-5-methylhexanoic acid (20 gm, 0.10 moles) was charged into mixture of ethyl acetate:water (9:1, 100 ml) and heated under stirring at 60° C. for 1.5-2 hours. Reaction mixture was cooled to 6° C. under stirring. Solid was filtered, washed with ethyl acetate (5 ml) and dried under vacuum to give the title compound.

Yield: 16 g (80%)

HPLC: 99.21%

### Example 3

### Preparation of Pregabalin

(R)(−)-3-(carbamoylmethyl)-5-methylhexanoic acid (15 g, 0.080 moles) was charged into water (21 ml) under stirring and cooled to 10° C. Sodium hydroxide solution (49.7% w/v, 21.9 gm, 0.272 moles) was added to the reaction mixture at 10° C. Sodium hypochlorite (52.2 g, 0.84 moles) was added to the reaction mixture and stirred at 10° C. for 10 minutes. The reaction mixture was heated to 47° C. and stirred for 1.5-2 hours. After the completion of reaction, the reaction mass was cooled to 20° C., pH was adjusted to 2.5 by adding concentrated hydrochloric acid (30 ml). Sodium hydroxide solution (10 ml) was added to the reaction mass to adjust the pH to 5.5 under stirring at 20° C. The solid was filtered, washed with isopropanol (30 ml) and dried under vacuum to give the title compound.

Yield:10.53 g (83%)

HPLC: 97.57%

### Example 4

### Purification of Pregabalin

Crude pregabalin (10 g, 0.018 moles) was charged into a mixture of isopropanol:water (0.4:0.6, 9.5 ml) and stirred at room temperature. Sodium hydroxide (0.25 g, 0.0018 moles) was added to the reaction mixture and stirred at 75° C. for 4 hours under stirring. The reaction mixture was filtered over 0.45 micron filter paper at 75° C. and washed with mixture of isopropanol:water (0.4:0.6, 5 ml) and dried under vacuum to give the title compound.

Yield: 7.6 g (76%)

HPLC: 99.91%

### Example 5

### Preparation of Crude Pregabalin

R-CMHA (20 gm, 0.107 mol) was added to water (30 ml) and cooled to 5-10° C. To the resulting thick suspension was added 50% w/w aqueous sodium hydroxide solution (20.4 gm in 20 ml water, 0.257 mol, 2.4 molar eq) dropwise over a period of 25-35 minutes maintaining the temperature at 5-10° C. The reaction mixture stirred for 30 minutes. 12% w/w aqueous sodium hypochlorite solution (69.7 gm, 0.112 mol) was added drop wise to reaction mixture at 5-10° C. The reaction mixture was allowed to warm up slowly and was later heated to a temperature of 45° C. and stirred for one hour. The completion of the reaction was confirmed by TLC and cooled the mass to RT.

Impurity content (by HPLC) at RRT 0.51: 0.39% w/w.

The above reaction mixture was divided into two parts (Part A and Part B)

### Part A: Isolation of Crude Pregabalin (With Out pH Adjustment of 2-3)

The pH of reaction mixture was adjusted to 5.0-5.5 with aqueous sodium hydroxide, cooled to 5-10° C. and stirred for one hour. The solid was filtered and washed with 40 ml water and dried to give pregabalin.

Impurity content (by HPLC) at RRT 0.51: 0.14% w/w.

Purification of Crude Pregabalin Obtained from Part A:

The crude pregabalin was added to a mixture of water (47 ml) and isopropanol (32 ml), heated to 70-75° C. till complete dissolution of the material The resulting solution was filtered to remove insoluble particles at 70-75° C. The filtrate was cooled to 5-10° C. and stirred for one hour. The solid was filtered and washed with 15 ml of isopropanol and dried at 50° C. under vacuum to obtain pregabalin.

Impurity content (by HPLC) at RRT 0.51: 0.14% w/w

### Part B: Isolation of Crude Pregabalin (With pH Adjustment of 2-3)

To the reaction mixture at 25-35° C. was added 35% w/w aqueous hydrochloric acid (34 gm) to get the pH of mass to 2-2.5 and stirred for 20-30 minutes. 50% w/v aqueous sodium hydroxide solution (5 gm) was added to the reaction mixture to achieve pH 5.0-5.5. A peach colored suspension was obtained which was cooled to 5-10° C. and stirred for one hour. The solid was filtered and washed with 40 ml water and dried to give pale orange colored crude pregabalin.

Impurity content (by HPLC) at RRT 0.51: Not Detected

Purification of Crude Pregabalin Obtained from Part B:

The crude pregabalin was added to a mixture of water (47 ml) and isopropanol (32 ml), heated to 70-75° C. till complete dissolution of the material. The resulting solution was filtered to remove insoluble particles at 70-75° C. The solution was cooled to 5-10° C. and stirred for one hour. The solid was filtered and washed with 15 ml of isopropanol and dried at 50° C. under vacuum to give pregabalin.

Impurity content (by HPLC) at RRT 0.51 (by HPLC): Not Detected.

We claim:

1. A process for preparation of (R)(−)-3-(carbamoylmethyl)-5-methylhexanoic acid (R-CMHA) of formula (II) or its pharmaceutically acceptable salts, which comprises:
a) reacting an ester of formula (III) with ammonia reagent in presence of a lewis acid and a suitable solvent; 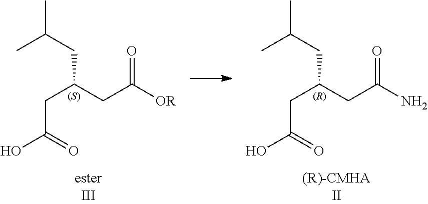
wherein R═C₁-C₆ alkyl, aryl, alkyl aryl
b) optionally isolating compound of formula (II);
c) optionally converting compound of formula (II) to its pharmaceutically acceptable salts;
d) optionally purifying compound of formula (II) or its pharmaceutically acceptable salts.

2. The ammonia reagent according to step a) of claim 1 is selected from NH₃ gas, (NH₃)₂CO₃, HCONH₂, Aq.NH₃, NH₂CONH₂, HCOONH₂, NH₄Cl.

3. The lewis acid according to step a) of claim 1 is selected from calcium chloride, lithium chloride, cerium chloride, Indium chloride, magnesium chloride, magnesium bromide, ammonium chloride, zinc chloride, boron trifluoride dietherate complex.

4. The solvent according to step a) of claim 1 is selected from alcohol solvents, aliphatic or alicyclic hydrocarbon solvents, halogenated hydrocarbon solvents, aromatic hydrocarbon solvents, or any mixtures thereof.

5. A process for preparation of pregabalin which comprises:
a) reacting an ester of formula (III) with ammonia reagent in presence of a lewis acid and a suitable solvent to provide (R)(−)-3-(carbamoylmethyl)-5-methylhexanoic acid (R-CMHA) of formula (II) or its pharmaceutically acceptable salts;
b) reacting (R)(−)-3-(carbamoylmethyl)-5-methylhexanoic acid (R-CMHA) of formula (II) or its pharmaceutically acceptable salts with a hypohalite in presence of suitable base and a solvent to provide pregabalin of formula (I) 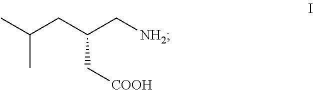
c) isolating pregabalin of formula (I)
d) optionally purifying pregabalin in a suitable solvent.

6. The hypohalite according to step b) of claim 5 is selected from sodium hypochlorite, sodium hypobromite, calcium hypochlorite, calcium hypobromite.

7. The base according to step b) of claim 5 is selected from alkali metal hydroxide, alkaline earth metal hydroxide, alkali metal carbonate, alkaline earth metal carbonate, alkali metal bicarbonates.

8. The solvent according to step b) of claim 5 is selected from water, alcohol, aliphatic or alicyclic hydrocarbon, halogenated hydrocarbon, aromatic hydrocarbon or any mixtures thereof.

9. A process for preparation of pregabalin substantially free of 4-ene impurity of formula IV, 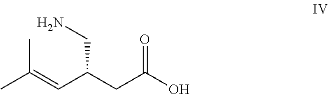
which comprises
a) reacting (R)(−)-3-(carbamoylmethyl)-5-methylhexanoic acid (R-CMHA) or a salt of formula II with hypochlorite in presence of suitable base and solvent; 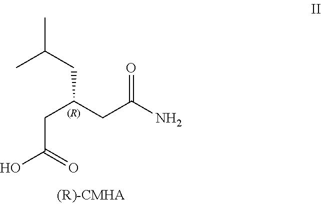
b) treating the reaction mass with an acid to get pH about 2.0-3.0;
c) treating with suitable base to result the pH of mass about 5.0-5.5;
d) isolating of pregabalin of formula (I) 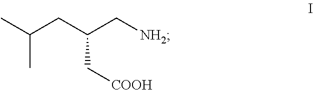
e) optionally purifying pregabalin in a suitable solvent.

10. Pharmaceutical compositions comprising pregabalin of formula (I) 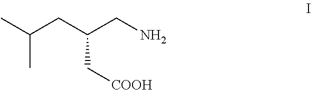
or its pharmaceutically acceptable salts prepared according to claim 5 together with one or more pharmaceutically acceptable excipient, carrier and diluents.

11. Pharmaceutical compositions comprising pregabalin of formula (I) 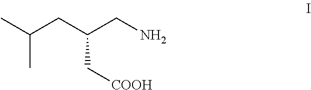
or its pharmaceutically acceptable salts prepared according to claim 9 together with one or more pharmaceutically acceptable excipient, carrier and diluents.
